(19) United States
(12) Patent Application Publication
(10) Pub. No.: US 2004/0102757 A1
(43) Pub. Date: May 27, 2004

(54) Disposable absorbent article with an elastic waistband and easy tear side seams

(75) Inventor: Christopher P. Olson, Neenah, WI (US)

Correspondence Address: STEPHEN E. BONDURA, ESQ., DORITY & MANNING, P.A., P.O. BOX 1449, GREENVILLE, SC, 29602-1449, US

(73) Assignee: KIMBERLY-CLARK WORLDWIDE, INC.

(21) Appl. No.: 10/301,878

(22) Filed: Nov. 21, 2002

(51) Int. Cl. A61F 13/15

(52) U.S. Cl. 604/396

(57) ABSTRACT

A disposable absorbent pant article, and method for making such article are provided. The article includes a chassis wherein facings of front and back panels are bonded together at side seams so as to define a waist opening and a pair of leg openings in the article. A waistband structure is attached to an edge portion of the front and back panels along a periphery of the waist opening and facings of the waistband structure are bonded with the front and back panel facings at the side seams. The waistband facings material is formed of a different material having a lower melting point as compared to the material of the front and back panel facings. In this manner, the waistband facings generally do not increase or decrease bond strength at the side seams.

### TECHNICAL FIELD OF THE INVENTION

The present invention relates generally to the field of disposable absorbent articles, for example children's training pants, and more particularly to a process for making such articles having an elastic waistband.

### BACKGROUND

Current disposable absorbent training pants for children going through the potty training stage have proved to be a particularly desirable and useful product. This is especially true for the child, when he or she has outgrown, or believe they have outgrown, diapers. Diapers are perceived by children as being for babies, and most children do not like being identified with or as babies. Thus, these children do not want to wear diapers, and instead prefer to wear a training pant that looks like adult underwear.

From a product fit, comfort, and performance aspect, elastic waistbands are a desirable feature for many types of training pants. Various designs of such elastic waistbands have been used, such as a single wide elastic member or a plurality of narrow elastic members. The waistbands may fully or partially surround the waist opening. Generally, the elastic waistbands are incorporated by one of two methods. The first method incorporates the elastic waistbands when they are in an extended, tensioned state. The second method incorporates the elastic waistbands while they are in a relaxed, untensioned state. The latter method may require the use of a special elastic material, such as a heat-elasticizable material.

With certain types of training pants, a tearable side seam is provided between the front and back panels of the garment. These seams are disposed so as to run along the outer sides of the child's leg and provide a quick and relatively easy means for a parent, or the child, to remove the article, particularly when the pants have been soiled and it is impractical to remove the pants by pulling the article down and off of the child's legs. However, the desirability of providing a “substantial” waistband to such tearable seam pants with a thickness and feel that is noticeably different from the outer cover and side panels of the pants can be problematic, particularly at the seam zones. The waistbands tend to add significant “strength” to the seams and it may be difficult for certain consumers to tear through the waistbands when trying to remove the pants.

The present invention provides a method for producing tearable side seam training pants having a substantial elastic waistband that does not interfere with tearing the seams and removing the pants.

### SUMMARY OF THE INVENTION

Objects and advantages of the invention will be set forth in part in the following description, or may be obvious from the description, or may be learned through practice of the invention.

The invention will be described and referenced herein as it relates to a process for making children's training pants. However, it should be understood that this is for illustrative purposes only. The invention is not limited to training pants, but relates to any type of absorbent disposable garment or article incorporating tearable side seams. Such articles may include, for example, incontinence articles, pull-up diapers, feminine hygiene products, swim pants, and the like.

A method according to the invention is provided for making an absorbent pant article, such as children's training pants, having tearable side seams and an elastic waistband. The waistband provides all of the beneficial aspects of a full (completely encircling) substantial waistband but does not detract from the ability to remove the article from a wearer by tearing the article along side seams thereof.

Initially, an article or “chassis” is provided having a front panel, a back panel, a crotch panel extending between the front and back panels, and an absorbent structure carried in the chassis. The front panel and back panels have longitudinal edge portions and lateral sides. Conventional processes for defining these type of initial articles in an in-line absorbent article manufacturing process are well known in the art. An elastic waistband structure is attached, for example by thermal bonding or with an adhesive, to longitudinally opposed waist edges of the front panel and back panel. A pant structure is then defined by joining together the sides of the front panel and the back panel at side seams to form a pair of leg openings and a waist opening.

In one embodiment, the facing portions of the front panel material and back panel material joined at the side seams are made of a first material (or layers of different materials) having a first melting point. Facing portions of the material (or layers of different materials) of the waistband structure joined at the side seams are made of a second material having a second lower melting point as compared to that of the front and back panel facing portions.

The step of joining the front and back panels may include bonding the panels along the side seams at a temperature and/or pressure selected so as to achieve a desired bond strength between the facing materials of the front and back panels having the first higher melting point and also at a temperature and/or pressure high enough to essentially thermally degrade the materials of the waistband facings having the second lower melting point. The waistband materials are degraded to such an extent that such materials do not form any sort of appreciable seam that requires additional force to tear above that required to tear the side seams of the front and back panels. In other words, the waistband materials do not add to or decrease from the strength of the side seam. The side seams thus may, if desired, have a generally uniform tear strength from the waist opening, through the waistband, and to the leg openings.

It should be appreciated that the method and articles according to the invention are not limited to any particular material or combination of materials, so long as the waistband materials can be thermally degraded while maintaining the strength and integrity of the side seams between the front and back panels. Also, it is to be understood that the style, configuration, or features of the absorbent article are not limiting characteristics of the invention. The invention has utility and usefulness in any application of disposable absorbent article wherein a waistband is a desired feature.

The invention will be desired in greater detail below through reference to embodiments illustrated in the figures.

### BRIEF DESCRIPTION OF THE FIGURES

FIG. 1 is a perspective view of a child's training pant incorporating principles of the present invention.

FIG. 2 is a cross-sectional view of the training pant of FIG. 1 taken along the lines indicated in FIG. 1.

FIG. 3 is a cross-sectional view of the training pant of FIG. 1 taken along the lines indicated in FIG. 1.

FIG. 4 is a schematic illustration of a method for making an elastic waistband for incorporation with an absorbent article according to the invention.

FIG. 5 is a schematic illustration of a method for making an absorbent article according to the invention.

FIGS. 6, 7, and 8 are illustrations relating to a test procedure described herein.

### DETAILED DESCRIPTION

The invention will now be described in detail with reference to particular embodiments thereof. The embodiments are provided by way of explanation of the invention, and not meant as a limitation of the invention. For example, features described or illustrated as part of one embodiment may be used with another embodiment to yield still a further embodiment. It is intended that the present invention include these and other modifications and variations as come within the scope and spirit of the invention.

The present invention provides an improved method for making absorbent articles, such as a child's training pant, having an elastic waistband wherein the waistband does not detract from the ability to remove the article from the wearer by tearing or separating the article along side seams. Principles of the invention will be described by reference to a child's training pant. However, as mentioned, it should be understood that the invention is not limited to training pants, but relates to any type of absorbent disposable garment or article incorporating tearable side seams. Such articles may include, for example, incontinence articles, pull-up diapers, and the like.

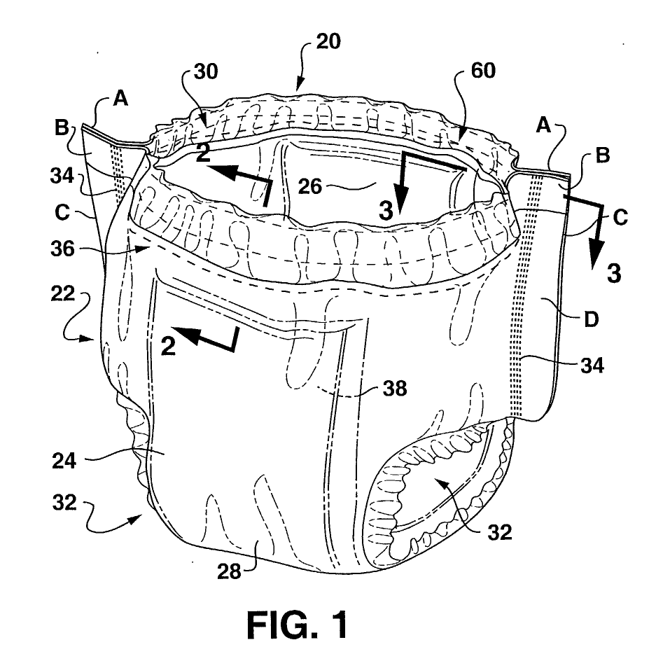

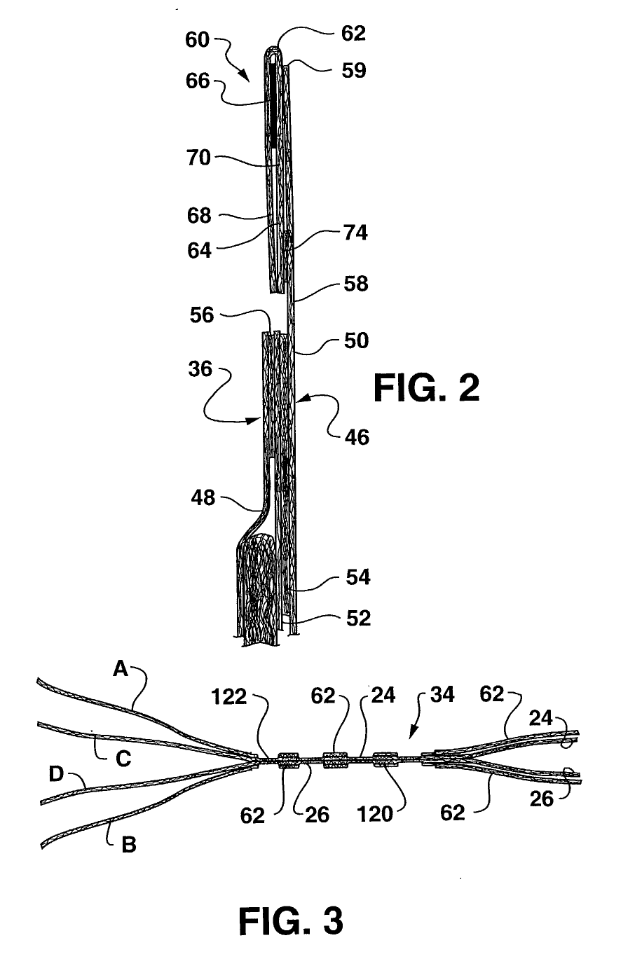

With reference to FIGS. 1 and 2, a disposable child's absorbent training pant 20 is illustrated. The pant 20 includes a chassis 22 having a front panel 24, a back panel 26, and a crotch panel 28 extending between the front and back panels. A waist opening 30 and a pair of leg openings 32 are formed by selectively joining the front panel 24 and back panel 26 at side seams 34. The side seams 34 extend between the waist opening 30 and respective leg openings 32. A waist border 36 peripherally surrounds the waist opening 30, and is formed upon joining the front panel 24 and the back panel 26 at seams 34. The side seams 34 will be discussed in greater detail below.

Still referring to FIGS. 1 and 2, the pant chassis 22 includes an absorbent structure 38 disposed in the crotch panel 28 and extending into the front panel 24 and back panel 26. In general, the absorbent structure 38 has a length dimension that is greater than the width dimension. As illustrated, the absorbent structure 38 may extend generally from the front panel waist border to the back panel waist border. The absorbent structure may have any suitable shape and configuration, as recognized in the art. It should be appreciated that the configuration of the absorbent structure 38 is not a particularly distinguishing feature of the invention.

The chassis 22 further includes an outer cover layer 46 and a liner 48. The absorbent structure 38 is sandwiched between the outer cover layer 46 and liner 48. The liner 48 is desirably a single layer of liquid permeable material, but may also include other layers of material. The outer cover layer 46 is desirably a two-layer material that includes an outer layer 50, which may be made of a non-woven liquid permeable material, and an inner layer 52, which may be made of a liquid impermeable material. The outer layer 50 and inner layer 52 may be joined together in any suitable manner, such as by an adhesive 54. The adhesive 54 is illustrated in the figures as a generally continuous layer and is exaggerated for sake of illustration. It should be appreciated that the adhesive 54 may be deposited in any suitable pattern for providing adequate adhesion of the materials. The liner 48 is desirably joined to the outer cover layer 46 by an adhesive 56, thereby sandwiching the absorbent structure 38 therebetween. Again, the adhesive 56 is illustrated as a continuous layer and is exaggerated in the figure for sake of illustration.

Referring to FIG. 2 in particular, the waist border 36 desirably includes an extension of one of the layers of the chassis 22. For example, the waist border 36 may be a multi-layer structure comprising the outer cover layer 46 and liner 48. For example, this extension may be an extension of the outer layer 50 of the cover layer 46, as illustrated in FIG. 2. This extension forms a peripheral edge portion 58 that surrounds the waist opening 30.

It should be appreciated that the description of the training pant 20 provided herein is for illustrative purposes only, and is not meant to limit the structure of a training pant 20 encompassed by the invention. The training pant 20 may have any one of a number of suitable designs or constructions. Examples of representative training pants are disclosed, for example, in U.S. Pat. No. 4,940,464, and U.S. Pat. No. 4,641,381, the disclosures of which are incorporated herein by reference for all purposes.

Continuing to refer to FIGS. 1 and 2, a separate elastic waistband system 60 is associated with the chassis 22 about the waist opening 30. The elastic waistband system 60 includes an elongate sleeve member 62 defining an elongate passage 64 therethrough. An elongate elastic member 66 is disposed in the passage 64. The sleeve member 62 may be formed from one layer of material, such as a non-woven liquid permeable material, by folding the material into a C-shaped configuration comprising an outer surface 68 and an inner surface 70, as particularly shown in FIG. 2. The outer surface 68 and inner surface 70 can be joined in any suitable manner, such as by an adhesive. The adhesive joins the outer and inner surfaces 68, 70 and the elongate elastic member 66 is substantially freely moveable within the elongate passage 64.

The elastic waistband system 60 may be joined to one of the layers comprising the chassis 22, such as the outer layer 50, in any number of ways. In the illustrated embodiment, the elongate sleeve member 62 is joined to the peripheral edge portion 58, as particularly shown in FIG. 2, such that the portion of the sleeve member 62 containing the elastic member 66 is generally coextensive with a waist edge 59 of the peripheral edge portion 58.

The elongate elastic member 66, freely moveable within the elongate passage 64, may comprise any manner of suitable elastic materials. Although illustrated in the figure as a single member of elastic material, the elongate elastic member 66 may comprise a plurality of elastic ribbons or strands. Because the elastic waistband system 60 is a separate structure from the chassis 22, the waistband system 60 can be constructed of different types of desired materials independent of the materials making up the chassis 22. This provides increased flexibility in the design and construction of the waistband elastic system 60, and allows for a selection of materials for forming the side seams 34 according to the invention.

Referring again to FIG. 1, the side seams 34 are formed by bonding the facings of the front panel 24 and back panel 26 and the material facings of the elongate sleeve member 62 such that a continuous side seam 34 is defined from the leg openings 32 to the periphery of the waist opening 30 defined by the outermost edge of the elastic waistband system 60. Thus, the upper portion of the side seams 34 will include joined flaps A and B (FIG. 3) of the elongate sleeve member 62, and joined flaps C and D of the front panel 24 and back panel 26.

The materials of the elongate sleeve member 62 are selected with respect to the material of the front and back panels 24, 26 such that when the side seams 34 are formed in a thermal bonding process (e.g., a process utilizing temperature and/or pressure to at least partially melt the materials to be bonded), the waistband structure 60 presents generally little or no additional structural integrity or strength to the seams 34 above that provided by the seam between the facings of the front and back panels. In other words, when removing the pant 20 from a wearer by grasping the pant either along the panels or waistband structure, and pulling or tearing the pant chassis along the seams 34, the waistband structure 60 does not appreciably add to the tearing force necessary to separate the seam 34. As discussed, a substantial waistband structure 60 is a desirable feature in many types of absorbent pant articles. With conventional articles, the waistband structures may have presented a problem in that they made it difficult to tear the pant 20 along the seams 34. This problem is not present in the structure described herein.

It should be appreciated that the invention is not limited to any particular bonding process, and may include for example any thermal process utilizing heat, laser, pressure, temperature, ultrasonics, or welding.

According to an aspect of the present invention, the materials selected for the elongate sleeve member 62, or other structure defining the waistband 60, has a lower thermal melting point as compared to the material of the front and back panels 24, 26. In this way, when forming the seams 34, by any appropriate thermal bonding process, such as ultrasonic bonding, any combination of the bonding temperature, energy, pressure, and dwell time can be controlled so as to form an adequate bond between the higher melt point facings C and D of the front and back panels 24, 26 while essentially thermally destroying or degrading the lower melt point facings A and B of the waistband structure 60. The lower melt point facings A and B are “degraded” or “destroyed” to the extent that essentially only a residue of the materials remains after the thermal bonding process. This residue has virtually no structural integrity and does not add to the strength of the seams 34. It may also be that the material is essentially vaporized, or is thinned-out to such an extent as to be negligible. The lower melt point material may also adhere to and be removed by the bonder. The “degradation” feature is conceptually illustrated in FIG. 3 wherein the seam 34 includes bonded points 122 interspaced between unbonded points 120. At the bonded points 122, the material of the flaps A and B is essentially nonexistent, and the bond is formed between the material of the flaps C and D. At the unbonded points 120, the material of the flaps A and B may still be present, but the materials are not bonded at these locations and, thus, the material flaps A and B do not add to the bond strength.

The components of the training pant 20 can be made of any suitable material or combination of materials well known in the field of personal care absorbent articles. For example, the absorbent structure 38 can comprise any suitable absorbent material, natural or synthetic, or a combination thereof, along with a super absorbent material. The absorbent material may also be encased in a tissue wrap (not shown) in order to maintain the integrity of the absorbent material. Suitable super absorbent materials are available from various vendors, such as Stockhausen GmbH & Co. of Germany; and Dow Chemical Company of Midland, Mich., U.S.A. Typically, the super absorbent material is capable of absorbing at least about 15 times its weight in water, and desirably is capable of absorbing more than about 25 times its weight in water. A suitable natural absorbent material is a wood pulp fluff identified by the trade designation CR1654 from U.S. Alliance of Childersburg, Ala., U.S.A. This particular wood pulp fluff is a bleached, highly absorbent sulfate wood pulp fluff containing soft wood fibers.

The outer cover layer 46 may be a single layer of a liquid permeable or liquid impermeable material, and may or may not have breathability, i.e. be vapor permeable. In a particular embodiment, the outer cover layer 46 is a two-layer composite comprising outer layer 50 and inner layer 52. The outer layer 50 may be a liquid permeable non-woven web, for example a spunbond bicomponent web or a bonded carded bicomponent web. Alternatively, the outer layer 50 may be a liquid permeable spunbond polypropylene non-woven web. The inner layer 52 may be a polyethylene film.

The liner 48 may be a liquid permeable and substantially hydrophobic material, such as a spunbond web, meltblown web, bonded carded web of synthetic polymer filaments, or combined synthetic filaments with natural fibers, such as rayon. Suitable synthetic polymers include, by way of example, polyethylene, polypropylene, and polyester. Liner 48 typically has a pore size that readily allows the passage of liquids, such as urine and other body exudates. If desired, the liner 48 may be treated with a surfactant to selectively adjust its degree of wetability, and can also be selectively embossed or perforated with discrete slits or holes.

All of the described adhesives, such as adhesives 54, 56, and 74, can be any adhesive suitable for joining the identified materials. Suitable adhesives can be obtained, for example, from Findley Adhesives, Inc., of Wauwatosa, Wis., or from National Starch and Chemical Company of Bridgewater, N.J. The adhesives can be applied in any manner such as by spraying, slot-coat extrusion, printing, or the like. The applied adhesive can be in any desired configuration, such as continuous or discontinuous beads, continuous or discontinuous swirls, meltblown patters, spray patterns, or the like.

The elongate sleeve member 62 of the waistband elastic system 60 may be a non-woven web or film, the only requirement being that the material have a lower thermal melting point than that of the material forming the side panels of the pant 20. In one particular embodiment, the elongate sleeve member 62 is formed of a 0.6 mil polyethylene film.

The elastic member 66 is desirably made of natural rubber, or an elastomeric material such as isoprene obtainable from JPS Elastomerics Company of Holyoke, Me. As mentioned, the elastic member 66 may be a single ribbon of material or a plurality of strands or ribbons of elastic material. A particularly desired material for use as a plurality of strands of elastic material is Lycra® 940 from EI DuPont de Nemours Company of Wilmington, Delaware.

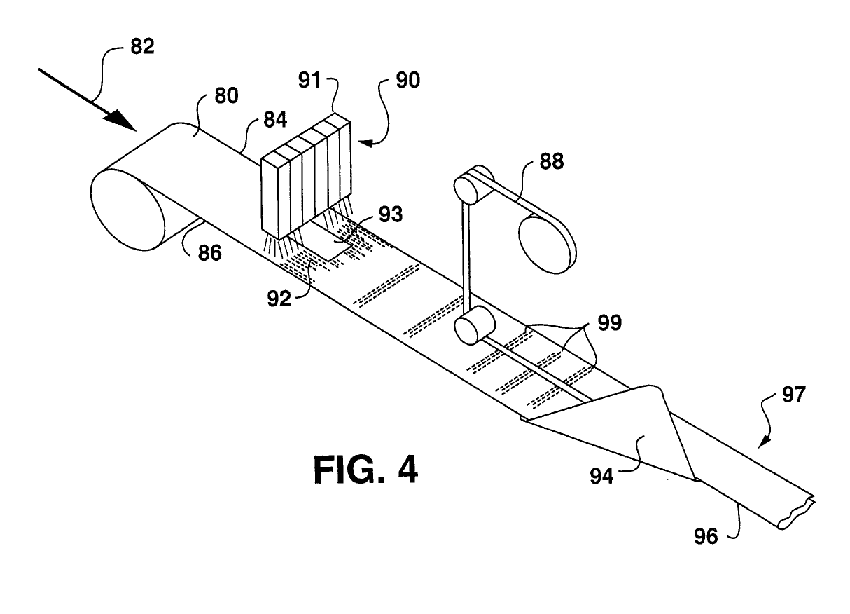

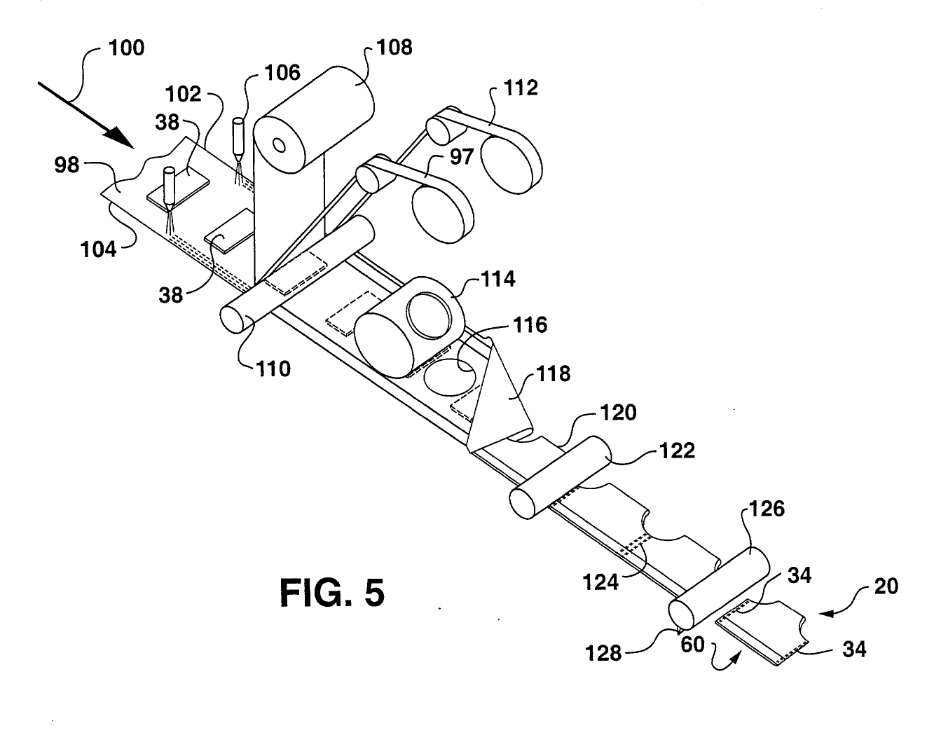

Referring now to FIGS. 4 and 5, an exemplary conceptual method is described relating to a manufacturing assembling line for making disposable absorbent training pants 20 according to one embodiment of the invention. Referring to FIG. 4, a first layer 80 of material having opposite edge portions 84, 86 is continuously moved in a first direction 82. The first layer 80 can be supplied in any suitable manner well known in the art, and subsequently will form part of the elongate sleeve member 62. An elongate elastic member 88 is continuously applied or provided in the first direction 82 in any suitable manner known in the art in a selectively tensioned state to the first layer 80. The elongate elastic member 88 will subsequently form part of the elongate elastic member 66. The elongate elastic member 88 can also be continuously applied or provided in a substantially untensioned manner, and, if so, it may be a specific type of elastomeric material commonly referred to as a heat-elasticizable material. This latter type of elastomeric material can be treated, such as by heat, to recover its latent elasticity. Generally, the elongate elastic member 88 will be joined to the first layer 80, prior to the folding of the first layer 80, by a pulsed adhesive system 90 for providing a predetermined adhesive pattern on the first layer 80 by selectively controlling a bank of spray nozzles 91. The adhesive may be sprayed or applied in a continuous pattern or an intermittent pattern. For example, the pattern may be applied in an adhesive zone 92 having a window 93 that is essentially void of adhesive. Alternatively, the adhesive may be applied in adhesive zones 99 which are intermittently and closely spaced together.

After providing the elongate elastic member 88 to the first layer 80, the first layer 80 is passed through a folding board 94, which continuously folds the first layer 80 in a direction generally transverse to the first direction 82 along a fold line 96 and over the elastic member 88. After folding the first layer 80, the elongate elastic member 88 is intermittently joined to the first layer 80 thereby resulting in a first elastic component 97, which will ultimately form the elongate sleeve member 62.

Referring to FIG. 5, a second elastic composite 112 can be made in a separate manufacturing assembly line in the same manner as the first elastic composite 97. After the first and second elastic composites 97, 112 have been made, they may be individually wound on rolls, and transported to another assembly line, such as the line illustrated in FIG. 5, for subsequent handling.

Referring now to FIG. 5, a base layer 98 having opposite edge portions 104 and 102 is continuously moved in a machine-direction 100. The base layer 98 may be a single layer of material, or a laminate or composite comprising, for example, the two layers that ultimately form outer layer 50 and inner layer 52 of the outer cover 46. The base layer 98 may also be made of a material suitable for use as liner 48. A pair of adhesive applicators, such as adhesive spray nozzles 106, apply adhesives, such as adhesive 74 (FIG. 2), along opposite edge portions 102, 104.

A plurality of absorbent structures 38 are registered on top of the base layer 98 at equally distantly spaced apart locations between the opposite edge portions 102, 104. The absorbent structures 38 are positioned on the base layer 98 such that their respective lengths are transverse to the machine-direction 100. The absorbent structures 38 can be provided in any suitable manner known in the art.

A top layer 108 is continuously supplied on top of the absorbent structures 38 and base layer 98. Just as the base layer 98 may be made of a material or layers of materials suitable for outer cover 46 or liner 48, the top layer 108 may also be made of materials suitable for use as the outer cover layer 46 or liner 48. In this particular description, the top layer 108 corresponds to the liner 48. The first elastic composite 97 and second elastic composite 112 are continuously delivered to the base layer 98 so as to be positioned on the base layer 98 along the respective edge portions 104, 102, and are joined to the base layer 98 by the adhesive supplied from the adhesive nozzles 106 (adhesive 74 in FIG. 2). A pressure roller 110 presses the elastic composites 97, 112, base layer 98, and top layer 108 together to assist in joining the layers together. The top layer 108 may be smaller in transverse width as compared to the base layer 98, and thus may not be in contact with the elastic composites 97, 112. The elastic composites 97, 112 will form the elongate sleeve member 62 (FIG. 2).

It should be appreciated that the elastic composites 97, 112 may be joined to either side of the base layer 98. For example, FIG. 5 illustrates the elastic composites 97, 112 joined on the same side of the base layer 98 on which the absorbent structures 38 are placed. If desired, the elastic composites 97, 112 can be joined on the opposite side of the base layer 98 such that the elongate sleeve member would be disposed on the opposite side of the outer cover layer 46.

A patterned rotary die, such as a pattern cutting roll 114 illustrated in FIG. 5, cuts a plurality of openings 116 through the top layer 108 and base layer 98, between the absorbent structures 38. The openings 116 will subsequently form the leg openings 32. If desired, the openings 116 can be formed by other means, such as by water-jet cutters, and may be cut into any desired form.

Thereafter, a folding board 118 folds the base layer 98 along a fold line 120 that is parallel to the machine-direction 100. A rotary ultrasonic bonder 122 or other suitable thermal bonding mechanism or device bonds the folded base layer 98 along a plurality of bond lines 124, which are generally transverse to the machine-direction 100. The bonding process along the bond lines 124 forms the seams 34, and may be continuous or intermittent along one or a plurality of lines. The bond lines 124 are located between the absorbent structures 38, and if desired can simultaneously bond each elongate elastic member 88 (FIG. 4) to its respective layer 80. As discussed, the parameters of the ultrasonic bonder 122, or other thermal bonding device, are set such that an adequate bond strength is formed between the material flaps C and D of the front and back panels 24, 26 (FIG. 1) while the lower melt point flaps A, B of the waistband structure 60 are disintegrated or thermally degraded and do not add to the strength of the seam 34.

A cutting roll 126 having a blade 128 cuts the base layer 98 along cut lines that are transverse to the machine-direction 100 and between the absorbent structures 38. The cut lines are located within a central region or area of the respective bond lines 124, thereby splitting a single bond 124 into two bond lines. The cutting of the base layer 98 results in a plurality of disposable absorbent training pants 20 having elastic waistband systems 60 and leg openings 32 formed by seams 34.

In the above-described process, the elastic composites 97, 112 are material independent of the chassis 22. Thus, the materials forming the elastic composites 97 and 112, particularly the material of the elongate sleeve member 62, may be selected independent of the materials of the pant chassis 22. Accordingly, it should be appreciated, that a vast number of combinations of materials are suitable for use in methods according to the invention wherein the material of the waistband structure has a lower thermal melting point than the material of the front and back panels. The invention thus is not limited to any particular combination of materials, and it is well within the ability of one skilled in the art to select suitable materials for achieving the side seams 34 described herein, as well as other desirable characteristics.

A brief discussion of “melting points” for polymers will aid in an appreciation of the present invention. In typical polymers used in the construction of disposable articles, the molecular weight of the material is not constant throughout. There may also be crystalline and amorphous regions within any given molecular chain. Molecular weight and the amount of crystallization all influence the temperature at which melting (phase change from solid to liquid) occurs. The melt point variability is compounded by the fact that polymers are poor conductors of heat so phase change phenomena may be localized. The melting point of a polymer may thus more properly be defined as a melting range. The melting properties may be described by specifying a 5% onset of melting (5% by weight of the material has changed from solid to liquid state) and a peak melt temperature (at least half of the material has changed from solid to liquid).

For purposes of this invention, the melt points of the polymer materials may be based on the temperature measurement method of ASTM D 3418-99. particularly sections 10.1 through 10.1.2 for measuring the peak temperature for the preliminary thermal cycle. The cooling and reheating cycles described in the ASTM are not relevant since in most ultra sonic bonding processes, the materials are only heated once. The preliminary thermal highest peak melt temperature is of primary importance for purposes of the present invention. The melting extrapolated onset temperature is of interest because, dependant on the compressive force used, the anvil embossing pattern, and other variables, the commingling and viscous flow of the materials can start at about this temperature.

With the factors discussed above affecting the melting points of various polymers (e.g., crystalline structure, film stress, plasticizers, impurities, and the like), it should thus be appreciated that the highest peak melting point of a polymer may vary. For some typical polymeric materials used in absorbent articles, the following estimates of the highest peak melt temperatures are believed accurate:

Isotactic polypropylene film: 160-165 degrees Celcius

Quenched Isotactic polypropylene film: 155-160 degrees Celcius

Syndiotactic polyporpylene: 135 degrees Celcius

Isotatic polyethylene: 121 degrees Celcius

A desired delta melting temperature (“delta T”) between the polymer materials is dependent on several variables, the most significant being the amount of time the materials are compressed, energized, and allowed to heat up in the bonding process. If the time is short, as it is in high speed continuous bonding processes, it is desirable to have a high delta T to maximize the flow of the lower peak melt materials out of the welded areas within the time available. The lower the viscosity of the material, the faster the material flows. Also, for a greater pressure, a greater amount of material flows. The pattern and configuration of the anvil also have an effect. With a dot-pattern weld area, the lower peak melt material need only flow a minimal distance to clear the weld spots between the two facings of the higher peak melt material. Factors that play a role in any sonic bonding process include: anvil pattern, nip pressure, frequency of sound (type of energy), amplitude of the sound (amount of energy), basis weights of the materials, addition of solvents to the materials, and the like. A desired delta T should take these factors into consideration. A delta T of at least 35 degrees Celcius may be a safe benchmark in a high speed continuous rotary bonding process to cover a wide range of the variables just discussed. Lower delta T's are possible if the variability factors are controlled and optimized. The following delta T's are non-limiting suggestions for particular combinations of materials:

Syndiotactic polypropylene/isotatic polyethylene: greater than 10 degrees Celcius

Quenched Isotactic polypropylene film/syndiotactic polypropylene: greater than 20 degrees Celcius.

Isotactic polypropylene film/Isotatic polyethylene: greater than 35 degrees Celcius.

Ultrasonic bonds or welds involve the melting and subsequent co-mingling of polyerms. An ultrasonic bonding process relevant to the present invention involves five general steps:

1. Compression: The materials are brought together under a suitable pressure. The amount of pressure is dependent on a number of variables such as th etype of bonding equipment, speed at which the work pieces go thorugh the bonder, composition of the materials being bonded, and the like. The optimal amount of compression can be determined by a reasonable amount of experimentation and is routine in the sep up of an ultrasonic bonding process. A rotary ultra sonic bonder is one dxample where a rotary anvil with an embossed pattern and a rotary sonic horn form a nip through which th e web that is beign bonded is pulled. In a plunge bonder, the materials to be bonded are placed on an anvil and a sonic horn is brought down on top of and compresses the materials, is then energized, and transfers energy to the materials.

2. Excitation: In an ultra sonic bonding process, sound waves excite the molecules and cause them to vibrate. The vibration causes heat which raises the temperature of the materials. Alternatively, the work piece could be heated by the transfer of heat energy from heated anvils in a plunge bonding process. Another alternative is to pre-energize the work piece with microwave radiation, or the like.

3. Viscous Flow of Lower Peak Melt Point Material(s): The lower melt point materials melt first and flow out of the area to be welded because of the pressure due to the compression against the anvil.

4. Viscous Flow of Higher Peak Melt Point Materials: The phase significantly overlaps with the previous phase. At the instant the lower melt point materials have been pushed aside in the areas of highest pressure (high points on the anvil pattern) and the higher melt point layers flow and co-mingle with each other. This flowing and co-mingling largely occurs in the temperature range between the 5% melt onset and the peak melt temperature.

5. Re-solidification: After the materials pass through the bonder or the energy is turned off, the materials re-solidify. The relatively high peak melt to high peak melt layers bond in the higher bonding pressure areas forming an area of consistent/predictable weld strength. The lower peak melt temperature materials pushed out of the target welded area also re-solidify in the regions between and around the high points on the anvil pattern. Thus, these materials do not affect the properties of the weld in the target area.

### Test Procedure

This procedure is a tensile strength bench test to measure the force required to separate a bonded seam that joins two materials. The materials can be composites or laminates of multiple components. The bonded seam can be formed from a pattern of bond points or small bonded regions. The force of separation is measured by determining load values as the two materials are pulled apart perpendicular to their plane of contact. The test values are an indication of how strongly the materials are adhered together, and how difficult it would be for a consumer to separate the layers when incorporated into a product, such as the side seam of a disposable training pant garment. The sample is pulled in the tensile tester until the sample pulls apart. Bond strength is the peak load result.

1. Overview

A material sample of two material layers joined by a bond such as an ultrasonic bond is assembled. The sample is prepared by aligning and bonding the materials together. Alternatively, the sample is cut out of a product with the ultrasonic seam in the middle of the cut strip. The sample can be cut from the waist band or the leg band region of the product depending on the purpose of the test. The sample is then placed between clamps on a tensile tester. One piece of material is held in the upper clamp, while the other is held in the lower clamp. The bond is arrayed between the clamps, approximately parallel to the edges of the clamp faces. The width of all materials to be tested is 1 inch (25.4 mm). The gage length is 2 inches (50.8 mm) between the edges of the clamp faces. The term “load” refers to the gram value measured by the load cells in the tensile tester.

The jaws are separated at a controlled rate until the bond is pulled apart. The load values generated on the material throughout this process are recorded. The peak load value is recorded as the bond strength.

Peak load values for samples of non-standard widths should be normalized by multiplying or dividing by the factor by which the sample width deviates from 1 inch (25.4 mm). For example, the peak load value derived by pulling apart a 0.5 inch (12.7 mm) wide sample should be multiplied by 2.

Suitable materials include side panel and/or waistband materials, which may comprise or be attached to materials used to form the disposable garments described herein.

2. Apparatus and Materials

* Constant Rate of Extension (CRE) tensile tester: such as an MTS tensile tester model Synergie 200 Test Bed; available from MTS® Systems Corporation, Research Triangle Park, N.C. USA.

* Load cells: A suitable cell selected so the majority of the peak load values fall between 10 and 90% of the manufacturer's recommended ranges of load cell's full scale value; for example, Model 100N available from MTS® Systems Corporation, Research Triangle Park, N.C. USA.

* Operating software and data acquisition system: such as MTS TestWorks® for Windows software version 4; available from MTS® Systems Corporation, Research Triangle Park, N.C. USA.

* Grips: pneumatic-action grips, top and bottom, identified as part number 38.00716 available from MTS Systems Corporation.

* Grip faces: 25 by 75-mm (1 by 3-inch) interlocking faces such as are available from MTS Systems Corporation.

3. Conditioning

Reasonable ambient conditions are required for testing. The instruments used should be calibrated as described in the manufacturer's instructions for each instrument.

4. Test Specimen

Specimens may be assembled from raw materials or removed from intact products. Several types of specimens can exist, a waist region specimen, a control (mid-seam) region specimen, a leg band specimen, and the like. For a given side panel composition, the control specimen is compared to any waist or leg region specimens that are prepared with that side panel composition.

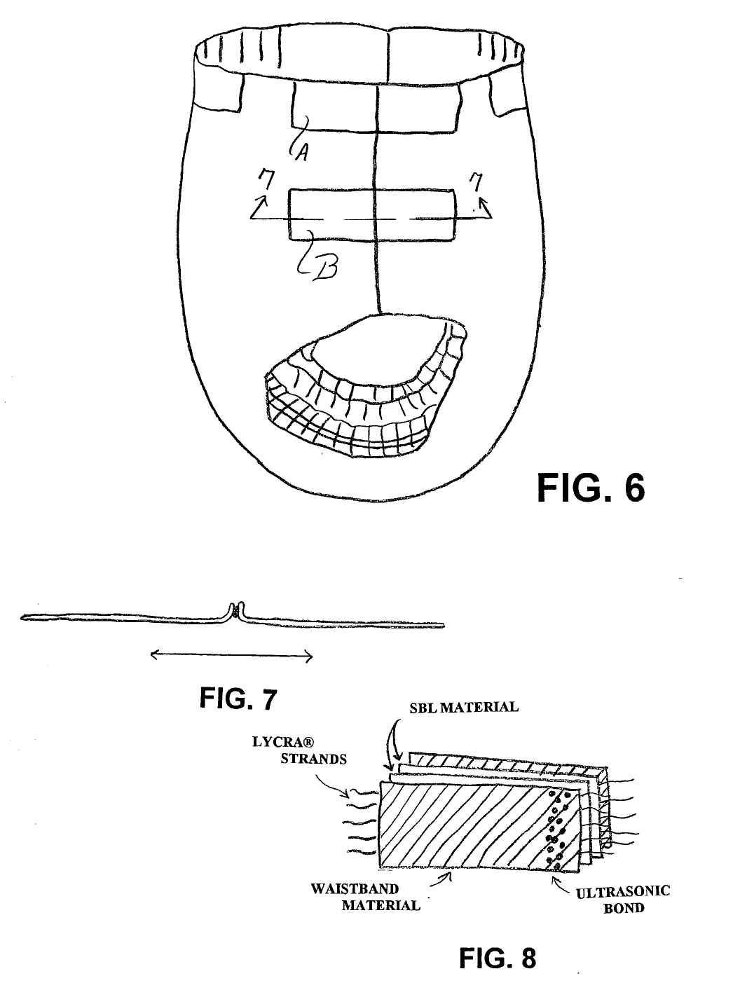

Specimens from products: A waist region specimen is taken by removing a 1 inch by 3 inch rectangular piece, in which the bond is centered in and perpendicular to the 3 inch dimension. The long dimension of the specimen should correspond to the circumferential dimension of the garment. The waist region specimen should comprise the top 1 inch of the bond in the pant, adjoining the waist opening (region A in test FIG. 6). A control region specimen is taken by removing a 1 inch by 3 inch rectangular piece, in which the bond is again centered in and perpendicular to the 3 inch dimension and the long dimension is oriented circumferentially in the garment. The control region specimen should be taken from a point approximately centered in the length of the bond (region B in test FIG. 6); in other words, the specimen should not include any waist or leg elastic. The control region specimen should not be taken from the region of the bond adjoining the leg opening, as this may possess leg-encircling elastic regions that may skew tensile results. Each pant type that is measured should be sampled at least at both the waist and central-bond regions; two specimen data sets (A and B) should be generated, corresponding to these regions.

Specimens from raw materials: Raw material specimens may be assembled by combining materials that would serve as side panel and/or waist elastics in a garment. Specimens using only side panel materials are regarded as controls, while specimens with both side panel and waist/leg elastic structures are regarded as tests (the latter type of structure is referred to as a “composite sample/specimen” below to reflect the addition of a waist/leg elastic component). Each material should be cut to at least 1 inch by 3 inches, with the long dimension corresponding to the circumferential dimension of a garment that would be made therefrom. Materials are then joined in a manner such as that described below, trimmed to have a final dimension of 1 inch by 3 inches, and tested as described.

Specimens for a control sample (i.e., lacking a waist elastic feature) may be prepared by bonding material for the front panel to material for the back panel. A corresponding waist region sample, comprising a set of “composite specimens,” may be prepared using the same combination of side panel materials, as well as any waist elastic structure that may be chosen for testing. Each component should be sized to provide a final (trimmed) sample dimension of 1 inch by 3 inches, in which the long dimension represents the circumferential dimension of a garment that would be made therefrom. The selected materials are combined in the appropriate orientation, bonded under the same conditions as the control specimen, trimmed to the specified dimensions, and tested as described.

The specimen is tested using the tensile test procedure that follows; the specimen is tested along the direction indicated by the arrow in test FIG. 7. At least four specimens of each sample should be tested, and the results averaged. The average value for the control of a given sample should be compared to the average value for each waist region specimen set of that sample.

5. Procedure

Tensile Tester test conditions

| Break sensitivity | 60% | |
|---|---|---|
| Preload? | No | |
| Test speed | 500 | mm/min |
| Gage length: | 2 | inches |
| | (50.8 | mm) |
| Number of cycles: | 1 | |

A. Using the tensile frame pushbutton controls for crosshead position, move grips to provide a gage length of 2 inches (50.8 mm). Take the crosshead channel to this initial gage length.

B. Place a material specimen so that the bond is centered (vertically) between the grips, held in a centered position (horizontally) within each grip, and oriented correctly (3 inch/76.2 mm dimension running the width direction on the grips). The specimen's vertical edges should be perpendicular to the nearest edges of the grip faces, and the bond should be parallel to the edges of the faces. The arrow in test FIG. 7 shows the direction in which the sample is pulled during the test.

C. Close the upper grips on the specimen and tare the load channel.

D. Hold the specimen in such a way as to minimize slack in the specimen, but do not place the specimen under tension, and close the lower grips on the specimen.

E. Run the test using the above parameters by clicking on the RUN button.

F. When the test is complete, save the data to a sample file.

G. Remove the specimen from the grips.

H. Run additional specimens of a given sample using steps B-E and G; the data for all specimens within a sample should be saved to a single file.

I. Continue testing all samples in this manner.

J. Data are reported as the average peak load value for each sample.

### Samples

Samples of various waistband configurations were prepared in the laboratory and tested using the above procedure. Two low melt point configurations (samples 1 and 2) were tested, as well as five high melt point configurations. These waistband structures (described below) were attached on top of stretch bond laminate (SBL) material, such as that used in side panels of HUGGIES® PULL-UPS® Disposable Training Pants. This material is a laminate of 18.5 gsm Krayton G2740 and 0.40 osy polypropylene spunbond facing. The retracted laminate basis weight at 150% stretch to stop is 3.36 osy. One edge of each waistband was aligned with an edge of the underlying SBL material, to provide a composite sample of a waistband structure with no overhang or projection beyond the SBL (see test FIG. 8). Composite samples were prepared using the following materials and methods.

Low melt point configurations:

Sample 1: A single layer of 0.6 mil (15.2 microns) polyethylene film with five strands of Lycra® elastic, available from E. I. DuPont de Nemours and Company, Wilmington, Del. U.S.A., attached using double sided adhesive such as Adhesive Transfer Tape 9509 available from Minnesota Mining and Manufacturing Company, St. Paul, Minn., U.S.A.

Sample 2: Four layers of 0.6 mil polyethylene film with five strands of Lycra® elastic, assembled as described in Sample 1.

High melt point configurations:

Samples 3 and 7: A laminate of 0.4 ounce per square yard (osy) (13.6 gram per square meter (gsm)) polypropylene spunbond (SB) with 23.5 gsm KRATON® elastomer available from Shell Chemical Company; the laminate being formed of two layers of spunbond in the configuration SB/KRATON/SB; and five strands of Lycra elastic adhered to the laminate using a construction adhesive such as H2525A, available from Findley Adhesives, Inc., of Wauwatosa, Wis., U.S.A.

Sample 4: A SB/Lycra/SB laminate formed using the spunbond described above, five strands of Lycra, and construction adhesive described above.

Sample 5: A film/Lycra/SB laminate formed using the polyethylene film and spunbond described above, and five strands of Lycra with the construction adhesive described above; the SB side of the laminate was placed against the SBL material when the composite sample was formed.

Sample 6: The same laminate as in Sample 5, but the film side of the laminate was placed against the SBL material when the composite sample was formed.

Bonding:

Samples 1-6 were bonded to SBL side panel material to form composite samples using an ultrasonic bonder 920 iw available from Branson Ultrasonics Corp. of Danbury, Conn., U.S.A. Bonder settings were as follows:

| | | |
|---|---|---|
| Air pressure | 60-70 | psi (pounds per square inch) |
| Weld time | 0.15 | sec |
| Hold time | 0.10 | sec |
| AB time | 0.25 | sec |
| AB delay | 0 | sec |

Composite samples were constructed to have two pieces of SBL material as inner layers, and two pieces of waist band material as outer layers (FIG. 8; Lycra strands shown for reference only). Pieces were aligned as shown and attached using double sided adhesive as described above (although adhesive is optional). Composite samples were bonded as shown and trimmed to 1 inch bonded length if necessary prior to testing.

Sample 7 was bonded using an in-line rotary ultrasonic bonder such as is used to form side panel bonds on HUGGIES® PULL-UPS® Disposable Training Pants.

The bond pattern used for all samples was comprised of four rows of pins, each pin being 0.036 inches (0.91 mm) in diameter. The outer three rows of pins (next to the edge of the material) has a pin-to-pin spacing (center to center) of 0.062 inches (1.57 mm), while the innermost row had a spacing of 0.125 inches (3.17 mm). The rows were spaced 0.052 inches (1.32 mm) apart, and offset successively 0.016 inches (0.41 mm) from one row to the next. Other suitable bond patterns that provide adequate bond strength may be used.

The strength of the seams was then tested at the waistband section of the seams to determine the breaking strength of the waistband side seam bond when a load is applied at a constant rate in a direction perpendicular to the direction of the seam, the “strength” being the maximum load (in kilograms) achieved before the seam ruptures. The results were compared to the strength of the same type of seams on side panels without a waistband. The results are provided summarized in the table below:

TABLE

| | Average Bond | Standard | Coefficient |
|---|---|---|---|
| Samples | Strength (kg) | Deviation | of Variability |
| Control | 2.3 | 0.43 | 19% |
| (no waistband) | | | |
| 1 | 2.0 | 0.36 | 18% |
| 2 | 2.1 | 0.13 | 6% |
| 3 | 3.5 | 0.78 | 23% |
| 4 | 4.6 | 0.52 | 11% |
| 5 | 3.2 | 0.24 | 8% |
| 6 | 3.0 | 0.68 | 22% |
| 7 | 1.0 | 0.84 | 82% |

The seam strength of the side panels without a waistband was measured at 2.3 kg. The seam strengths for the one-inch waistband samples with low melt point waistband facings (samples 1 and 2) were 2.0 kg and 2.1 kg, respectively. Thus, it can be seen that the low melt point waistband structures did not add to the bond strength of the underlying seam. The high melt point waistband structures (samples 3 through 6) significantly added to the bond strength at the waistband sections of the seams.

It should be appreciated that the seam strength was measured just at the one-inch wide waistband structure, and not along the entire side seam. The principle here being that, for the higher melt point samples, the additional layers of waistband material at the same or a higher melt point as compared to the side panels will require more bonder energy than that required to bond just the side panels alone. Thus, if the bonder energy is optimized for the waistband section, the energy may be so high as to damage the side panel seams below the waistband, thus yielding an insufficient bond in the seam below the waistband. Similarly, if the bonder energy is optimized for the side panel seams, such energy may not be high enough to melt all of the layers of the waistband, resulting in an insufficient waistband seam.

Sample 7 emphasizes the point just discussed. This sample was conducted on training pants run on a “pilot line” using a continuous commercial type rotary bonder process that was set up to optimize the strength of the side panel material. However, when the one-inch waistband was added, the bonder energy was insufficient to properly melt the extra layers of material, resulting in an undesirably low (1.0 kg) strength in the waistband area.

The bonder parameters can be set so that the anvil essentially cuts through the lower melt point materials in the waistband with minimal energy loss and properly melts the underlying side panel materials to form a bond of desired strength. A desired bond strength is between about 2 kg to about 6 kg, and desirably about 5 kg. The settings for particular types of bonders in combination with particular combinations of materials can be determined so that a desired overall bond strength is achieved between the side panel facings while degrading the waistband facings.

It should be understood that resort may be had to various other embodiments, modifications, and equivalents to the embodiments of the invention described herein which, after reading the description of the invention herein, may suggest themselves to those skilled in the art without departing from the scope and spirit of the present invention.

What is claimed is:

1. A method for making an absorbent pant article having tearable side seams and an elastic waistband, comprising:
providing an article having a front panel, a back panel, a crotch panel, and an absorbent structure carried in the article, the front panel and back panel having longitudinal waist edge portions and lateral sides;
attaching an elastic waistband structure to the waist edge portions of the front panel and back panel;
defining a pant chassis by joining together the sides of the front panel and the back panel at side seams to form a waist opening defined by the opposed edge portions of the front and back panels and a pair of leg openings, wherein facing portions of the material of the front panel and back panel joined at the side seams are formed of a first material having a first melting point, and facing portions of the material of the waistband structure joined at the side seams are formed of a second material having a second lower melting point; and
wherein said step of joining the front and back panels includes thermally bonding the panels along the side seams so as to achieve a desired bond strength between the facing materials of the front and back panels having the first higher melting point while essentially thermally degrading the materials of the waistband facings having the second lower melting point.

2. The method as in claim 1, wherein the joining step is carried out by an ultrasonic bonding process.

3. The method as in claim 1, wherein the absorbent pant article is child's training pants.

4. The method as in claim 1, wherein the waistband structure is provided as an elongate sleeve member having an elastic member disposed therein.

5. The method as in claim 1, wherein the material of the front and back panels having the first higher melt point is a polypropylene spunbond material.

6. The method as in claim 5, wherein the polypropylene spunbond material is a facing layer of a stretch bond laminate material.

7. The method as in claim 1, wherein the material of the waistband structure having the lower melt point is a polyethylene film.

8. A disposable absorbent pant article, comprising:
a chassis having a front panel, a back panel, and a crotch panel extending between said front and back panels, opposed facings of said front and back panels being bonded together at side seams of said chassis so as to form a pant structure having a waist opening and a pair of leg openings;
a waistband structure attached to a waist edge portion of said front and back panels along a periphery of said waist opening with opposed facings of said waistband structure being bonded with said front and back panel facings at said side seams, said waistband facings being formed of a different material from said front and back panel facings; and
wherein said waistband facings generally do not increase bond strength at said side seams.

9. The article as in claim 8, wherein said waistband facings are formed of a material having a lower melting point compared to a melting point of said front and back panel facings.

10. The article as in claim 9, wherein a delta between the lower and higher melting points is at least greater than about 10 degrees Celcius.

11. The article as in claim 9, wherein a delta between the lower and higher melting points is at least greater than about 20 degrees Celcius.

12. The article as in claim 9, wherein a delta between the lower and higher melting points is at least greater than about 35 degrees Celcius.

13. The article as in claim 9, wherein said side seams comprise a thermally bonded seam such that the bond strength of said side seams is determined by the bond strength of said front and back panel, said waistband facings being thermally degraded to such an extent that they do not appreciably increase bond strength therealong.

14. The article as in claim 13, wherein said waistband facings comprise a polyethylene film.

15. The article as in claim 14, wherein said waistband structure comprises a sleeve of said polyethylene film having an elastic member disposed therethrough.

16. The article as in claim 13, wherein said front and back panel facings comprise a stretch-bond-laminate material having a polypropylene spunbond facing layer.

17. The article as in claim 8, wherein said article is a child's training pants.

18. The article as in claim 8, wherein said bond strength of said side seams is between about 2 kilograms and about 6 kilograms.
